SEQUENCE LISTING (4 sequences)

SEQ ID NO: 1 - DNA, 1176 bp, Vitis vinifera
gagtctggtggagaattcgaattcgaattcatggagacttcaaaactttttcttctctcctcctctctcctcctagtcttactcgccactcgtccatgtccttctctctctgaacgttgcaacccaaaagacaaaaaagttctccttcaaatcaaaaaagccctagacaatccctacattctagcttcgtggaatcccaacaccgattgctgcggatggtactgcgtcgaatgtgacctcaccacccaccgcatcaactcgctcaccatcttctccggccagctatccggccagattcccgacgctgttggtgaccttccgttcctcgagaccctcatcttccgcaagctctctaacctcaccggtcagatcccgccggcgattgccaaactcaagcacctaaaaatggttcgccttagctggaccaacctctccggtcccgtgccggcgttcttcagcgagcttaagaacctcacgtacctcgacctctccttcaataacctatctggacccattcccggcagcctctctctcctccccaacctcggcgcactccatctcgaccggaaccacctcacaggcccaatccctgactccttcggaaaattcgccggctctaccccaggtctacacctctcacacaaccaactttccgggaaaatcccatattctttcagaggattcgaccccaatgtcatggacttatcgcgtaacaagcttgagggtgacctgtcaatattcttcaatgccaataagtcaacacagatcgttgacttctcacggaacttgttccagtttgatctttcgagagtggaattcccgaagagtttgacgtcgttggacctttcgcataacaagatcgccgggagcctgccggagatgatgacttctctggatttacagttcctgaacgtgagttacaatcgtttgtgtggtaagattccggtgggtgggaagttgcagagcttcgattacgactcctactttcacaatcggtgcttgtgtggtgctccactccagagctgcaagggtggtggtggttctggtggtggtggttctggtggtggtggttctgagtctggtggaagttcttttgattttatggatggttatgataagcctgtgaaagggagaaaaatcaattggatgaaagccggcatattagaatcagacagg

SEQ ID NO: 2 - DNA, 618 bp, Nelumbo nucifera
aagttcttttgattttattgatggttatgataagcctgtgaaagggagaaaaatcaattggatgaaagccggcatattagaatcagacagggtgttaactgtcagtccatactatgcagaagaacttgcttcaggcatagaaaaaggtgtggaactagataacataattcggaagactggcattactggtattgtgaatggcacagatgttcaggagtggaacccaaccacagacaaatatatcagtgttaaatatgatgctacaactgttatggatgcaaagcctcttctaaaggaagcacttcaatctgaagttgggttgcctgtggaccgaaatatccctgtaataggctttattggtagactcgaagagcagaaaggttcagatattcttgcagcatcaattcccaaattcattggagagaatgttcagataattgtcctcgggaccggtaaaaaggcctttgagaagcaacttgagcaactagagatcaaatatcctgacaaagccagaggagttgcaaaattcaatgttcctcttgcccatatgatcatagctggagctgactttctgctgatcccaagtagatttgaaccatgtggtcttattcagttaca

SEQ ID NO: 3 - DNA, 1731 bp, Artificial Sequence
  PGIP portion from Vitis vinifera, gtf1 portion from Nelumbo Nucifera
gagtctggtggagaattcgaattcgaattcatggagacttcaaaactttttcttctctcctcctctctcctcctagtcttactcgccactcgtccatgtccttctctctctgaacgttgcaacccaaaagacaaaaaagttctccttcaaatcaaaaaagccctagacaatccctacattctagcttcgtggaatcccaacaccgattgctgcggatggtactgcgtcgaatgtgacctcaccacccaccgcatcaactcgctcaccatcttctccggccagctatccggccagattcccgacgctgttggtgaccttccgttcctcgagaccctcatcttccgcaagctctctaacctcaccggtcagatcccgccggcgattgccaaactcaagcacctaaaaatggttcgccttagctggaccaacctctccggtcccgtgccggcgttcttcagcgagcttaagaacctcacgtacctcgacctctccttcaataacctatctggacccattcccggcagcctctctctcctccccaacctcggcgcactccatctcgaccggaaccacctcacaggcccaatccctgactccttcggaaaattcgccggctctaccccaggtctacacctctcacacaaccaactttccgggaaaatcccatattctttcagaggattcgaccccaatgtcatggacttatcgcgtaacaagcttgagggtgacctgtcaatattcttcaatgccaataagtcaacacagatcgttgacttctcacggaacttgttccagtttgatctttcgagagtggaattcccgaagagtttgacgtcgttggacctttcgcataacaagatcgccgggagcctgccggagatgatgacttctctggatttacagttcctgaacgtgagttacaatcgtttgtgtggtaagattccggtgggtgggaagttgcagagcttcgattacgactcctactttcacaatcggtgcttgtgtggtgctccactccagagctgcaagggtggtggtggttctggtggtggtggttctggtggtggtggttctgagtctggtggaagttcttttgattttatggatggttatgataagcctgtgaaagggagaaaaatcaattggatgaaagccggcatattagaatcagacagggtgttaactgtcagtccatactatgcagaagaacttgtttcaggcatagaaaaaggtgtggaactagataacgtaattcggaagactggcattactggtattgtgaatggcacggatgttcaggagtggaacccaaccacagacaaatatatcagtgttaaatatgatgctacaactgttatggatgcaaagcctcttctaaaggaagcacttcaagcagaagtcgggttgcctgtggaccgaaatatccctgtaataggctttattggtagactcgaagagcagaaaggttcagatattctcgcagcatcaattcccaaattcattggagagaatgttcagataattgtcctcgggaccggtaaaaaggcctttgagaagcaacttgagcaactagagatcaaatatcctgacaaagccagaggagttgcaaaattcaatgttcctcttgcccatatgatcatagctggagctgactttctgctgatcccaagtagatttgaaccatgtggtctcattcaattacatcaccaccatcatcattgatgaggtacc

SEQ ID NO: 4 - protein, 575 aa, Artificial Sequence
  PGIP portion from Vitis vinifera, gtf1 portion from Nelumbo Nucifera
ESGGEFEFEFMETSKLFLLSSSLLLVLLATRPCPSLSERCNPKDKKVLLQIKKALDNPYILASWNPNTDCCGWYCVECDLTTHRINSLTIFSGQLSGQIPDAVGDLPFLETLIFRKLSNLTGQIPPAIAKLKHLKMVRLSWTNLSGPVPAFFSELKNLTYLDLSFNNLSGPIPGSLSLLPNLGALHLDRNHLTGPIPDSFGKFAGSTPGLHLSHNQLSGKIPYSFRGFDPNVMDLSRNKLEGDLSIFFNANKSTQIVDFSRNLFQFDLSRVEFPKSLTSLDLSHNKIAGSLPEMMTSLDLQFLNVSYNRLCGKIPVGGKLQSFDYDSYFHNRCLCGAPLQSCKGGGGSGGGGSGGGGSESGGSSFDFMDGYDKPVKGRKINWMKAGILESDRVLTVSPYYAEELVSGIEKGVELDNVIRKTGITGIVNGTDVQEWNPTTDKYISVKYDATTVMDAKPLLKEALQAEVGLPVDRNIPVIGFIGRLEEQKGSDILAASIPKFIGENVQIIVLGTGKKAFEKQLEQLEIKYPDKARGVAKFNVPLAHMIIAGADFLLIPSRFEPCGLIQLHHHHHHGT

(19) United States
(12) Patent Application Publication
(10) Pub. No.: US 2023/0242921 A1
(43) Pub. Date: Aug. 3, 2023

(54) ANTIMICROBIAL PROTEIN AND RELATIVE USE

(71) Applicant: DELPHINUS BIOTECH S.R.L., Trento (IT)

(72) Inventors: Alessandro PAPARELLA, Marcon (VE) (IT); Marco CAPPELLARO, Pontecchio Polesine (RO) (IT); Enrico FIORE, Vigonza (PD) (IT)

(73) Assignee: DELPHINUS BIOTECH S.R.L., Trento (IT)

(21) Appl. No.: 17/910,791

(22) PCT Filed: Mar. 12, 2021
(86) PCT No.: PCT/IB2021/052079

§ 371 Date: Sep. 9, 2022

Related U.S. Application Data
(60) Provisional application No. 62/989,067, filed on Mar. 13, 2020.
(60) Provisional application No. 63/005,474, filed on Apr. 6, 2020.

(30) Foreign Application Priority Data
Feb. 5, 2021 (IT) 102021000002597

(51) Int. Cl. C12N 15/62; C12N 15/82; A01N 63/50; A01N 65/00

(52) U.S. Cl. CPC C12N 15/62; C12N 15/8279; A01N 63/50; A01N 65/00

(57) ABSTRACT

The invention relates to a synthetic fusion protein having high antimicrobial activity. In particular, the invention relates to the use of said protein to eliminate pathogenic microorganisms from environments and surfaces. In particular, the synthetic fusion protein object of the present invention can be used in the preparation of disinfectant solutions that can be used in the outpatient, hospital and domestic field, in high traffic environments, in filters of air inlet and extraction systems or air conditioners or air treatment unit ATU, in environments used for food preparation. In general, in all those areas where it is necessary to adopt antimicrobial prophylaxis directed against gram+bacteria, viruses, mycoplasma and fungi.

### TECHNICAL FIELD

The present invention relates to the production and use of a synthetic fusion protein having a high antimicrobial activity. The protein object invention can be used in various fields:
in environmental disinfection, the protein object of the present invention can be used in the preparation of disinfectant solutions that can be used in the clinic, hospital and domestic field, in high traffic environments, in the filters of air intake and extraction systems or air conditioners or ATU treatment units, in environments used for food preparation. In general, in all those areas where it is necessary to adopt antimicrobial prophylaxis.
in the medical, veterinary or cosmetic field alone or as a basis for antibacterial, antifungal, antiviral preparations or to combat mycoplasma infections and phytoplasma infestations, for topical, cutaneous or mucosal use, via aerosol. As an ingredient in mouthwashes, toothpastes and skin creams.
in the agricultural and phytopharmaceutical field, the protein according to the invention can be used for the same purpose to combat infections in plant species, both used as such and by means of the agro-infiltration technique.

### STATE OF THE ART

There are many bacteria and many viruses that have in common as an attacking mechanism the target biological structures, which are constituted by proteins with a structural function, located inside the plasma membrane in order to give it mechanical resistance. At the end of the interaction of the biochemical reaction between the two species, a permeation is obtained which ultimately leads to the destruction of the cell wall, through lysis. This biochemical action turns out to be a common character that requires the action of a single conserved mechanism present in the plant world by a class of enzymes: the polygalacturonases which are

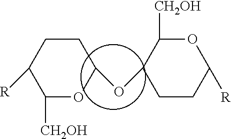

able to split the bond between the two heterocyclic rings of cellulose, through a endoglucosidase (cellulase) typical of bacteria and fungi with a lytic action against the infested plant tissue. In other cases, however, especially regarding virus attacks, the fusion of the biological structures that allows the transmission of the genetic material of the virus to the eukaryotic cell occurs through the combination of the Spikes by means of the membrane receptors allowing the introduction of the genetic material. Both of these mechanisms of infection require interaction with the different characteristics of the surface proteins that allow the pathogens to enter the vegetal eukaryotic or plant cells.

Polygalacturonases (PG) are cellulases, they are proteins belonging to the Bass of glycosylases, which catalyze the hydrolysis reaction: Polygalacturonic acid+H₂O [symbol] Polygalacturonic acid (broken)+Galacturonic acids. PG plays an essential role in the fruit ripening process.

During maturation the protopectins are degraded to pectic acids by the action of pectinesterase. Subsequently, the pectic acids, which are polymers of galacturonic acid, are hydrolyzed and made soluble by PG, with consequent softening of the pulp. Plant organisms have developed a defense system based on polygalacturonase inhibiting proteins (PGIPs) capable of inhibiting the pectinesterase reaction (Jaillon O, et al. Nature, 2007 Sep 27. PMID 17721507.

Among the various classes of PGIP, there is one with a degrading activity, composed of a series of repeated leucines that form the so-called “leucine zipper” that recognizes pectinesterases and degrades them by releasing hydrogen peroxide on contact. The ionic species released (H++O22−), come into close contact with the outer structures of the complex and begin to oxidize and reduce to transfer and acquisition of the outer layer electrons of the atoms, the glycoprotein components, coming in contact with the two ionic species above. Such leucine rich repeatitions (LRR) consist of 2-45 motifs of 20-30 amino acids in length which are generally bow-shaped arc or horseshoe [1].

The LRR can be found in proteins and come from eukaryotic viruses, which appear to provide a structural framework for the formation of protein-protein interactions [2, 3].

The analyzed sequences of the LRR protein group suggested the existence of different LRRs generating many subfamilies. The significance of this classification is that the repetitions of different subfamilies never occur simultaneously and most likely evolved independently, among the plant Phyla. However, it is now clear that all major LRR classes had curved horseshoe-shaped structures with a parallel beta sheet on the concave side and mainly helical elements on the convex side.

At least six LRR protein families characterized by different lengths and repeated consensus sequences have been identified.

Eleven segments of LRR residues (LxxLxLxxN/CxL), corresponding to the beta strand with adjacent ring regions, are conserved in the LRR proteins, while the remaining parts of the repeats (defined here as variables) can be very different. Some classes of PGIP take shape from a linear sequence of amino acids followed by two halves in “loops” to form a horseshoe-like protein.

The concave face and adjacent loops are the most common protein interaction surfaces on LRR proteins. The 3D structure of some protein-ligand LRR complexes shows that the concave surface of the LRR domain is ideal for alpha-helix interaction, thus supporting earlier conclusions that the elongated and curved LRR structure provides an outstanding framework for achieving diverse protein-protein interactions [2].

The prediction of the molecular computational model suggests that the conserved model LXXLxL, which is shorter than the previously proposed LxxLxLxxN/CxL is sufficient to impart the characteristic horseshoe, with protein curvature with repetitions of 20 to 30 residues [5].

The subfamily of PGIP named above with defensive functions is divided into two classes: cytoplasmic or anchored to the bacterial membrane. The latter has a portion of myristic acid which allows it to anchor to cell membranes and thus oriented it will go directly into contact with the exogenous glucan and/or peptido-glucan structures typical of fungal bacteria and viruses. This contact triggers a redox reaction breaking the bonds of these structures destroying the infesting microorganism [6].

Glycosyl transferases are enzymes that catalyze the transfer of sugar fractions from activated donor molecules to specific acceptor molecules, forming glycosidic bonds. Glycosyl transferases can be classified as retention or inversion enzymes based on the stereochemistry of substrates and reaction products [Sinnott, M L (1990) Catalytic mechanisms of enzymatic glycosyl transfer. Chem. Rev. 90, 1171-1202]. The glycosyl transferase (a reductase called gtf1) coming from Nelumbo nucifera 5E9U_A has the generic function of making the biosynthesis of disaccharides, oligosaccharides and polysaccharides.

Coronaviruses are a class of viruses identified in the 1960s, and initially described as viruses capable of causing common colds.

SARS CoV-2 is in particular a virus of the SARS-related coronavirus/SARS-CoV species, belonging to the coronavirus family and having a viral genome consisting of a single RNA helix of about 30 kb. The virion has four structural proteins, known as: protein S (spike), E (envelope), M (membrane) and N (nudeocapsid); SARS-CoV-2 spike proteins are glycoproteins responsible for coronavirus entry into host cells and consist of two functional subunits, S1 and S2 subunits. The S1 subunit consists of the N-terminal domain (NTD) and the receptor binding domain (RBD). The function of the S1 subunit is to bind to the receptor on the host cell. The function of the S2 subunit is to fuse the membranes of viruses and host cells. The cleavage site at the boundary between S1 and S2 subunits is called the S1/S2 cleavage site for proteases.

This glyco-protein complex begins fusion with cell receptors and then establishes a fusion bond which ultimately results in the transmission of the genetic material within the eukaryotic cell and the consequent destruction of the capsid and the retro-transcription of the virus.

Given the economic importance, numerous products have been developed over the years that are able to counteract the attack of the aforementioned microorganisms.

There is a strong need for new methods to combat these types of infections which are increasingly problematic for managing the danger of transmissibility. The current use of antiseptic products capable of environmentally containing the spread of bacterial and/or viral loads is currently borne by very aggressive chemicals such as: Na+HCLO— (sodium hypochlorite), Benzalkonium chloride, or through glutarladehyde or substances such as some species of acids that contain an active free chlorine activity between 0.1% and 0.5%.

These chemical species with redox activity, however, have a short life, since by combining with atmospheric oxygen 02 they become inert quickly.

For Example with Hypochlorite:

HClO—+O₂ [symbol] H₂O+Cl—

the inertization reaction is the above.

The reaction that occurs shortly after if there are no biological structures to be reduced, and therefore the disinfection process in various capacities, including spraying of various types of surfaces, must be repeated. In the same way, any activity that requires a high density of people with a possibility of zonal contagion, must be subjected again to systematic and also systemic disinfection if it has a hydraulic system for air distribution with devices that must convey treating thousands of cubic meters per hour.

### SUMMARY

The subject of the present invention is a biological method for counteracting the attack and proliferation of pathogenic microorganisms (such as Gram+, mycoplasmas, phytoplasmas, microscopic fungi) and viruses such as SARS-Cov-2 (coronaviridae).

In fact, the subject of the invention is a synthetic protein made using gene sequences coding for the polygalacturonase inhibitor derived from Vitis vinifera and an active subunit of the glycosyl transferase (gtf1 reductase) coming from Nelumbo nucifera 5E9U_A which has proved surprisingly able to enhance the reducing effect by producing a phenomenon of adhesiveness on the bacterial/viral glycosidic surfaces thanks to its enzymatic activity.

Therefore, the subject of the present invention is a synthetic fusion protein encoded by the sequence having SEQ ID no 3 and having an amino acid sequence of SEQ ID no 4.

The nucleotide sequence of SEQ ID no. 3 and/or a sequence having at least 90% and more preferably 95% sequence identity with SEQ ID no. 3;

The present invention also relates to a protein having the amino acid sequence of SEQ ID 4 and/or a sequence having 90% and more preferably 95% sequence identity with SEQ ID no.4.

The present invention also relates to a synthetic fusion protein encoded by the sequence having SEQ ID no 3 and having sequence SEQ ID no 4 for use in the medical field.

In particular in the medical, veterinary or cosmetic field, alone or as a base for antiviral antifungal antibacterial preparations or to combat mycoplasma infections and phytoplasma infestations, for topical, cutaneous or mucosal use, via aerosol. As an ingredient in mouthwashes, toothpastes and skin creams.

The present invention also relates to a process for the production and purification of the synthetic fusion protein encoded by the sequence having SEQ ID no 3 and having sequence SEQ ID no 4.

Said process comprises the following fundamental steps:

I. In a vector of expression, comprising a selection marker, insert a nucleic acid comprising at least one of: a sequence of SEQ ID no 3, a sequence having at least 90% sequence identity with SEQ ID no. 3 and a sequence having at least 95% identity with SEQ ID no. 3;
II. using said vector for the transformation of competent cells suitable for the use of said vector;
III. select the competent cells transformed with said vector and multiply them in culture;
IV. perform a lysis of the competent cells of point III;
V. select and purify the protein according to the invention from the lysate obtained at point IV.

The present invention also relates to the synthetic fusion protein encoded by the sequence having SEQ ID no 3 and having sequence SEQ ID no 4 for the treatment of infections in plant species, preferably in agriculture and phytopharmaceuticals; the protein according to the invention can be used for the same purpose both used as such and by means of the agro infiltration technique with A. tumefaciens.

The present invention also relates to a method for the treatment of plant pathogens, preferably phytoplasmas and fungi, where said method comprises the application on the plant or on parts thereof of the protein according to the invention or of compositions comprising it.

The use of the synthetic fusion protein encoded by the sequence having SEQ ID no 3 and having sequence SEQ ID no 4 for the treatment of environments and surfaces is also an object of the present invention. The synthetic fusion protein object of the present invention can be used in the preparation of disinfectant solutions that can be used in the clinic, hospital and domestic field, in high traffic environments, in filters of air inlet and extraction systems or air conditioners or ATU, in environments used for food preparation.

In general, in all those areas where it is necessary to adopt antimicrobial prophylaxis.

The present invention also relates to a method for the control or elimination of viruses, gram+bacteria, mycoplasmas, phytoplasmas, microscopic spore and oospore fungi, preferably S. aureus and Sars-CoV-2 from environments or surfaces where this method comprises the application on the surface or on parts thereof of the protein according to the invention or of compositions comprising it.

The present invention also relates to a process for the destruction of glycoproteins included in viruses, gram+bacteria, mycoplasma and microscopic spore and oospore fungi.

The present invention also relates to a composition comprising said protein at different concentrations which constitutes an antimicrobial solution that can be used in various fields in environmental disinfection, both in humans and animals, as a base for antibacterial/antifungal preparations for topical, cutaneous or nasal mucus use.

Further objects and advantages will become apparent from the detailed description of the invention.

### BRIEF DESCRIPTION OF THE FIGURES

FIG. 1: an embodiment of the expression vectors according to the present invention. Highlighted in an oval, the positions of the restrictions encoding enzymes for the insertion of the sequence of interest PGIP-GTF1. In particular, vectors for expression in Pichia Pastoris and Bacillus subtilis are represented.

FIG. 2: Structure of Gtf1 from Nelumbo nucifera. The sub-unit selected and inserted in the fusion protein is highlighted.

FIG. 3: Panel A GEL SDS PAGE showing the presence of a purified protein after isolation on a purification column.

Panel B: immuno-trans blotting anti his-tag to highlight the presence and levels of the correctly cloned fusion protein. The highlighted band indicates the presence of the histidine tail bound to the PGIP+GTF1 fusion complex detected by immuno-blotting technique.

For both panels: 1 maker-2 control+albumin-3 run buffer negative control-4 sonicated cell pellet-5 Test sample purified with his tag column-6 markers

FIG. 4: PGIP+GTF1 bactericidal/virucidal efficacy levels, indicative of the effect observed under the microscope at a distance of 48 and 72 hours.

Panel A View under optical microscope of the absence of S. aureus bacteria after 48 h contact with fusion protein, peroxide bubbles released upon contact with the protein and biological structures

Panel B View under optical microscope of biological structures lysed at 72 h from the Biological recognition effect and H2O2 release

Panel C Optical microscope view of the S. aureus culture at time 0

FIG. 5: Immuno-blotting test to detect the spike protein of SARS Cov2 after contact with PGIP+GTF1 Ad: 1 h, 24 h, 48 h, with PGIP+GTF1.

Panel A without protease inhibitor,

Panel B with protease inhibitor.

For both panels: M Marker-1 Spike Sars Cov2 Protein-2 buffer-3 16 μl SEQ Protein Id 4-4 10 μl SEQ PGIP/GTF1 Protein Id 4-5 buffer-6 Spike Sars-Cov2 Protein-7 Spike Protein Sars-Cov2+PGIP/GTF1 Protein from SEQ Id 4-8 Spike Sars-Cov2 Protein-9 Spike Sars-Cov2 Protein+PGIP/GTF1 SEQ Protein Id. 4, 10 Spike Sars-Cov2 Protein 11 Spike Sars-Cov2 Protein+Protein PGIP/GTF1 SEQ Id 4

FIG. 6: Panels A,B,C,D show the agro-adhesion experiment in which the purified protein at 62 KDa weight PGIP/GTF1 according to the invention was sprayed (4 ml containing 400 μg mixed with 0.0005% non-ionic foliar adhesive) on the surface of Vitis vinifera leaves already contaminated by viticultural plasmopara respectively to:

Panel A: time 0

Panel B: 10 hours from contact

Panel C: 24 hours from contact

Panel D: 48 hours from contact with

FIG. 7: Structure of the PGIP/GTF1 fusion protein according to the invention.

FIG. 8: Aspergillus, in contact with 400 μg of raw extract, after 48 h of contact FIG. 8A and after 72 h 8B compared to the control FIG. 8C

FIG. 9: vector map of pRI 201 AN highlighting the two multiple MCS1 AND MCS2 Boning sites this type of vector allows double hyper-expression of the same PGIP+GTF1 gene thanks to the presence of a CAM 35S viral promoter and a NOSter terminator.

FIG. 10: A B.cinerea infection on leaf tissue. B effects of blocking the infection after 10 h from agroinfiltration by means of A. tumenfaciens modified by the vector pRI 201 AN which is transmitted in the lesion starting to synthesize PGIP+GTF1.

FIG. 11: Photo under optical microscope of the conditions at 72 hours of cell cultures subjected to cytotoxicity tests Panel A A2780 cells of human ovarian cancer Panel B MSTO-211 H cells of biphasic mesothelioma For both panels the experiments of: control (containing complete medium)-control+buffer-1 nM protein solution-2.5 nM protein solution-5 nM protein solution-10 nM protein solution-20 nM protein solution.

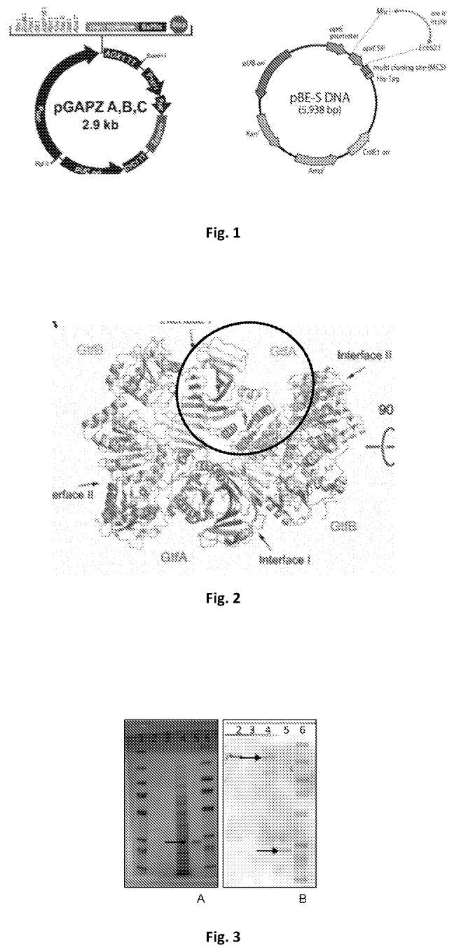

### DETAILED DESCRIPTION OF THE PREFERRED EMBODIMENTS

The present invention relates to a synthetic fusion protein, called PGIP+GTF1 or PGIP/GTF1, encoded by the sequence having SEQ ID no 3 and having an amino acid sequence of SEQ. ID no. 4 carried out using coding gene sequences:
for the polygalacturonase inhibitor derived from Vitis vinifera and
for an active subunit of glycosyl transferase (gtf1 reductase) from Nelumbo nucifera 5E9U_A which was surprisingly able to enhance the reducing effect by producing a phenomenon of adhesiveness on bacterial/viral glycosidic surfaces thanks to its enzymatic activity.

The object of the present invention is therefore a biological method for counteracting the attack and proliferation of viruses, gram+bacteria, mycoplasma and microscopic spore and oospore fungi. The gene sequences were selected after several pairing studies, compared and chosen for their intrinsic characteristics; in particular the PGIP sequence was selected according to what described in WO2019/077477 and the gtf1 subunit was selected by the inventors for its high enzymatic activity.

The literature shows that the selected sub-unit of the GTF1 glycosyl-transferase (reductase) according to the invention is able to recognize also the bacterial proteins belonging to the secA complex, secretory complex of Gram positive bacteria (Current Topics in microbiology and Immunology chapt.Protein and Sugar Export and Assembly in Grampositive Bacteria ed. Springer pag 45-67), and its presence increases the efficiency of the whole fusion protein complex thanks to the action of recognition and to bind this secA complex and by carrying out its lytic action, on the aforementioned glycoprotein complex to which it adheres.

The sequence listing titled “Sequence_Listing.txt,” which was created Mar. 2, 2023, and had a file size of 10 KB, is incorporated herein as if fully set forth.

SEQ ID NO 1:
sequence of PGIP, polygalacturanase inhibiting
protein from vitis vinifera.
GAGTCTGGTGGAGAATTCGAATTCGAATTCATGGAGACTTCAAAACTTT
TTCTTCTCTCCTCCTCTCTCCTCCTAGTCTTACTCGCCACTCGTCCATG
TCCTTCTCTCTCTGAACGTTGCAACCCAAAAGACAAAAAAGTTCTCCTT
CAAATCAAAAAAGCCCTAGACAATCCCTACATTCTAGCTTCGTGGAATC
CCAACACCGATTGCTGCGGATGGTACTGCGTCGAATGTGACCTCACCAC
CCACCGCATCAACTCGCTCACCATCTTCTCCGGCCAGCTATCCGGCCAG
ATTCCCGACGCTGTTGGTGACCTTCCGTTCCTCGAGACCCTCATCTTCC
GCAAGCTCTCTAACCTCACCGGTCAGATCCCGCCGGCGATTGCCAAACT
CAAGCACCTAAAAATGGTTCGCCTTAGCTGGACCAACCTCTCCGGTCCC
GTGCCGGCGTTCTTCAGCGAGCTTAAGAACCTCACGTACCTCGACCTCT
CCTTCAATAACCTATCTGGACCCATTCCCGGCAGCCTCTCTCTCCTCCC
CAACCTCGGCGCACTCCATCTCGACCGGAACCACCTCACAGGCCCAATC
CCTGACTCCTTCGGAAAATTCGCCGGCTCTACCCCAGGTCTACACCTCT
CACACAACCAACTTTCCGGGAAAATCCCATATTCTTTCAGAGGATTCGA
CCCCAATGTCATGGACTTATCGCGTAACAAGCTTGAGGGTGACCTGTCA
ATATTCTTCAATGCCAATAAGTCAACACAGATCGTTGACTTCTCACGGA
ACTTGTTCCAGTTTGATCTTTCGAGAGTGGAATTCCCGAAGAGTTTGAC
GTCGTTGGACCTTTCGCATAACAAGATCGCCGGGAGCCTGCCGGAGATG
ATGACTTCTCTGGATTTACAGTTCCTGAACGTGAGTTACAATCGTTTGT
GTGGTAAGATTCCGGTGGGTGGGAAGTTGCAGAGCTTCGATTACGACTC
CTACTTTCACAATCGGTGCTTGTGTGGTGCTCCACTCCAGAGCTGCAAG
GGTGGTGGTGGTTCTGGTGGTGGTGGTTCTGGTGGTGGTGGTTCTGAGT
CTGGTGGAAGTTCTTTTGATTTTATGGATGGTTATGATAAGCCTGTGAA
AGGGAGAAAAATCAATTGGATGAAAGCCGGCATATTAGAATCAGACAGG.
SEQ ID NO 2:
sequence of gtf1 from granule-bound starch
synthase from Nelumbo nucifera
AAGTTCTTTTGATTTTATTGATGGTTATGATAAGCCTGTGAAAGGGAGA
AAAATCAATTGGATGAAAGCCGGCATATTAGAATCAGACAGGGTGTTAA
CTGTCAGTCCATACTATGCAGAAGAACTTGCTTCAGGCATAGAAAAAGG
TGTGGAACTAGATAACATAATTCGGAAGACTGGCATTACTGGTATTGTG
AATGGCACAGATGTTCAGGAGTGGAACCCAACCACAGACAAATATATCA
GTGTTAAATATGATGCTACAACTGTTATGGATGCAAAGCCTCTTCTAAA
GGAAGCACTTCAATCTGAAGTTGGGTTGCCTGTGGACCGAAATATCCCT
GTAATAGGCTTTATTGGTAGACTCGAAGAGCAGAAAGGTTCAGATATTC
TTGCAGCATCAATTCCCAAATTCATTGGAGAGAATGTTCAGATAATTGT
CCTCGGGACCGGTAAAAAGGCCTTTGAGAAGCAACTTGAGCAACTAGAG
ATCAAATATCCTGACAAAGCCAGAGGAGTTGCAAAATTCAATGTTCCTC
TTGCCCATATGATCATAGCTGGAGCTGACTTTCTGCTGATCCCAAGTAG
ATTTGAACCATGTGGTCTTATTCAGTTACA.
SEQ ID no. 3:
nucleotide sequence of the PGIP + GTF1 fusion
protein according to the invention
GAGTCTGGTGGAGAATTCGAATTCGAATTCATGGAGACTTCAAAACTTT
TTCTTCTCTCCTCCTCTCTCCTCCTAGTCTTACTCGCCACTCGTCCATG
TCCTTCTCTCTCTGAACGTTGCAACCCAAAAGACAAAAAAGTTCTCCTT
CAAATCAAAAAAGCCCTAGACAATCCCTACATTCTAGCTTCGTGGAATC
CCAACACCGATTGCTGCGGATGGTACTGCGTCGAATGTGACCTCACCAC
CCACCGCATCAACTCGCTCACCATCTTCTCCGGCCAGCTATCCGGCCAG
ATTCCCGACGCTGTTGGTGACCTTCCGTTCCTCGAGACCCTCATCTTCC
GCAAGCTCTCTAACCTCACCGGTCAGATCCCGCCGGCGATTGCCAAACT
CAAGCACCTAAAAATGGTTCGCCTTAGCTGGACCAACCTCTCCGGTCCC
GTGCCGGCGTTCTTCAGCGAGCTTAAGAACCTCACGTACCTCGACCTCT
CCTTCAATAACCTATCTGGACCCATTCCCGGCAGCCTCTCTCTCCTCCC
CAACCTCGGCGCACTCCATCTCGACCGGAACCACCTCACAGGCCCAATC
CCTGACTCCTTCGGAAAATTCGCCGGCTCTACCCCAGGTCTACACCTCT
CACACAACCAACTTTCCGGGAAAATCCCATATTCTTTCAGAGGATTCGA
CCCCAATGTCATGGACTTATCGCGTAACAAGCTTGAGGGTGACCTGTCA
ATATTCTTCAATGCCAATAAGTCAACACAGATCGTTGACTTCTCACGGA
ACTTGTTCCAGTTTGATCTTTCGAGAGTGGAATTCCCGAAGAGTTTGAC
GTCGTTGGACCTTTCGCATAACAAGATCGCCGGGAGCCTGCCGGAGATG
ATGACTTCTCTGGATTTACAGTTCCTGAACGTGAGTTACAATCGTTTGT
GTGGTAAGATTCCGGTGGGTGGGAAGTTGCAGAGCTTCGATTACGACTC
CTACTTTCACAATCGGTGCTTGTGTGGTGCTCCACTCCAGAGCTGCAAG
GGTGGTGGTGGTTCTGGTGGTGGTGGTTCTGGTGGTGGTGGTTCTGAGT
CTGGTGGAAGTTCTTTTGATTTTATGGATGGTTATGATAAGCCTGTGAA
AGGGAGAAAAATCAATTGGATGAAAGCCGGCATATTAGAATCAGACAGG
GTGTTAACTGTCAGTCCATACTATGCAGAAGAACTTGTTTCAGGCATAG
AAAAAGGTGTGGAACTAGATAACGTAATTCGGAAGACTGGCATTACTGG
TATTGTGAATGGCACGGATGTTCAGGAGTGGAACCCAACCACAGACAAA
TATATCAGTGTTAAATATGATGCTACAACTGTTATGGATGCAAAGCCTC
TTCTAAAGGAAGCACTTCAAGCAGAAGTCGGGTTGCCTGTGGACCGAAA
TATCCCTGTAATAGGCTTTATTGGTAGACTCGAAGAGCAGAAAGGTTCA
GATATTCTCGCAGCATCAATTCCCAAATTCATTGGAGAGAATGTTCAGA
TAATTGTCCTCGGGACCGGTAAAAAGGCCTTTGAGAAGCAACTTGAGCA
ACTAGAGATCAAATATCCTGACAAAGCCAGAGGAGTTGCAAAATTCAAT
GTTCCTCTTGCCCATATGATCATAGCTGGAGCTGACTTTCTGCTGATCC
CAAGTAGATTTGAACCATGTGGTCTCATTCAATTACATCACCACCATCA
TCATTGATGAGGTACC.
SEQ ID NO 4:
Amino acid sequence of the fusion protein
according to the invention
ESGGEFEFEFMETSKLFLLSSSLLLVLLATRPCPSLSERCNPKDKKVLL
QIKKALDNPYILASWNPNTDCCGWYCVECDLTTHRINSLTIFSGQLSGQ
IPDAVGDLPFLETLIFRKLSNLTGQIPPAIAKLKHLKMVRLSWTNLSGP
VPAFFSELKNLTYLDLSFNNLSGPIPGSLSLLPNLGALHLDRNHLTGPI
PDSFGKFAGSTPGLHLSHNQLSGKIPYSFRGFDPNVMDLSRNKLEGDLS
IFFNANKSTQIVDFSRNLFQFDLSRVEFPKSLTSLDLSHNKIAGSLPEM
MTSLDLQFLNVSYNRLCGKIPVGGKLQSFDYDSYFHNRCLCGAPLQSCK
GGGGSGGGGSGGGGSESGGSSFDFMDGYDKPVKGRKINWMKAGILESDR
VLTVSPYYAEELVSGIEKGVELDNVIRKTGITGIVNGTDVQEWNPTTDK
YISVKYDATTVMDAKPLLKEALQAEVGLPVDRNIPVIGFIGRLEEQKGS
DILAASIPKFIGENVQIIVLGTGKKAFEKQLEQLEIKYPDKARGVAKFN
VPLAHMIIAGADFLLIPSRFEPCGLIQLHHHHHHGT.

The sequences according to the invention are in any case reported in the attached sequence listing.

Therefore, the nucleotide sequence of SEQ ID no. 3 and/or a sequence having at least 90% and more preferably 95% sequence identity with SEQ ID no. 3;

The present invention also relates to a protein having the amino acid sequence of SEQ ID 4 and/or a sequence having 90% and more preferably 95% sequence identity with SEQ ID no.4.

The present invention also relates to a synthetic fusion protein encoded by the sequence having SEQ ID no 3 and having an amino acid sequence of SEQ ID no 4.

The present invention also relates to a process for the production and purification of the synthetic fusion protein according to the invention.

Said process includes the following basic steps:

I. In an expression vector, comprising a selection marker, insert a nucleic acid comprising at least one of: a sequence of SEQ ID no 3, a sequence having at least 90% sequence identity with SEQ ID no. 3 and a sequence having at least 95% identity with SEQ ID no. 3;
II. using said vector for the transformation of competent cells suitable for the use of said vector;
III. select the competent cells transformed with said vector and multiply them in culture;
IV. perform a lysis of the competent cells of point III;
V. select and purify the protein according to the invention from the lysate obtained at point IV.

In one embodiment, in order to obtain expression and to be able to purify the protein according to the invention, the sequence having SEQ ID No. 3 was cloned into a vector for expression in bacteria or yeasts.

The vectors suitable for use according to the invention are known to those skilled in the art, in a preferred embodiment pBE-s DNA are preferably used, and secondly the vector pGAPZa-A (respectively sold by Takara and Thermofisher).

Various promoters known to the skilled in the art can be used to promote the transcription of the sequence according to the invention. In a preferred embodiment, the B. subtilis Secretory Protein Expression System (TAKARA) is used.

The protein according to the invention can be produced in various bacteria and yeasts suitable for the purpose and known to the skilled in the art, in a preferred embodiment the protein 701.1907 according to the invention is produced in Bacillus subtilis or Pichia Pastoris (Cregg et al., 1985; Cregg et al., 1989, Clare et al., 1991a; Clare et al., 1991b; Romanos et al., 1991) and during the transcription of the protein, glycosylation of the same leads to its natural form or hyperglycosylation it increases its metabolic capacity. In Pichia Pastoris the protein is post-translation hyper-glycosylated in 5 points, its molecular weight varies from 62 KDa to 200 KDa. In Bacillus subtilis, hyper-glycosylation brings the weight of the protein to 120 KDa. This hyper-glycosylation can be removed during purification procedures. In one embodiment, for example using the B. subtilis Secretory Protein Expression System (takara), in order to facilitate its purification, a tag sequence of 6 histidine residues (His-Tag). Other types of tags known to those skilled in the art are however suitable for the purpose according to the invention. To obtain the protein according to the invention, the method applied was the following:

1) RNA extraction from Vitis vinifera and Nelumbo nucifera
2) RT-PCR for the amplification of the gene portions of SEQ ID no 1 and 2, preferably performed using modified primers comprising sequences recognized by restriction enzymes (Gibson method) so as to provide the amplified with the desired sequence for subsequent digestion;
3) Following amplification, the PCR products are subjected to purification, first digestion in order to ligate the two fragments and subsequently to a ligase reaction aimed at obtaining a fragment comprising the sequence Id no 3.
4) Following the product of ligase obtained in point 3) is purified and subjected to a second digestion by insertion in a vector, preferably pBE-s DNA, and secondly in the vector pGAPZa-A, expressly selected for its ability to hyper-expression exclusively in bacteria and yeasts with non-protease.
5) The vectors comprising the sequence of SEQ Id no 3 are used to transform competent cells suitable for the purpose. Competent cells are preferably Pichia Pastoris or Bacillus subtilis. The transformation takes place preferably by electroporation since chemical transformation is not excluded.
6) The transformed cells are selected, preferably with antibiotics, and multiplied in a suitable medium known to the skilled in the art. LB/YPD is preferably used for Bacillus subtilis and Pichia Pastoris respectively.
7) After an adequate culture time selected on the basis of the microorganism, preferably 24 h-48 h, the cells are lysed to extract the total proteins and to proceed with the purification of the protein of interest preferably on an affinity column.

Optionally, the product obtained from the purification step is subjected to encapsulation in lipids, preferably phospholipids in order to facilitate its diffusion and protection from self-oxidation, thus extending the time of residence on the surfaces, and favoring the affinity with microbial structures.

Optionally, the product obtained from the purification step or from the encapsulation step is subjected to freeze-drying. In the context of the present invention, by raw or crude extract is meant the cellular lysate subjected to centrifugation and sonication but not to purification on an affinity column.

In one embodiment, the engineering of Pichia Pastoris and Bacillus subtilis was achieved by electroporation or by transformation of the bacteria competent to receive the plasmid (chemical method).

In one embodiment, said coding sequence for PGIP+GTF1 is cloned in 5′-3′ “IN FRAME” with the expression structure of the plasmids according to the invention.

In an alternative embodiment the ligated product obtained at point 3) is inserted in a vector suitable for use in A. Tumefaciens and subsequently in this electroporate; preferably the vector is the vector pRI-201AN (takara) which possesses two multiple Boning sites which therefore provides the vector with the ability to double express PGIP-GTF1, and the vector after purification is electroporated in the non-tumorigenic bacterium A. tumefaciens LB 4404 (takara). In order to verify the correct production of the protein according to the invention, the competent bacteria transformed with the vectors comprising the expression cassette of SEQ ID no. 3 capable of producing the protein according to the invention and labeled with 6 histidine tags, were lysed after the adequate culture time. Following the mechanical lysis which took place by sonication of 5 min′ at a frequency>20kH, on ice, the lysate was passed into the imidazole gradient purification column up to 100 mM to detach, after a first elution, the protein complex.

Different samples were run on SD-PAGE gels. In FIG. 3 it is in particular possible to observe how the supernatant of the sonicated cells, the pellet of the sonicated cells (or crude extract) at the concentration of 100 μg/μl (in lane 4) are run together with the positive control albumin (possessing the property of being detected with anti his-tag antibodies at 5 μg/μl) in lane 2 with the purified (lane 5). At the end of the electrophoretic run, the gel was subjected to western blot to transfer the proteins onto the nitrocellulose membrane. The membrane was incubated, with primary anti-His-tag antibody, and a secondary antibody labeled with peroxidase for detection of the protein in ECL. In panel 3 A (colored gel) it is possible to observe the presence in lane 5 of the band which highlights the presence of well-determined and over-expressed purifications with a molecular weight of 62 Kda (non-glycosylated form of PGIP/GTF1). In panel 3 B the immuno-blot reveals the presence in lane 4 (sonicated cell pellet not subjected to purification) of the overexpression of a highly glycosylated protein at 200 kd by weight; the purified protein in non-glycosylated form of 63 KDa is detected in lane 5b.

This experiment demonstrates that the fusion protein exists, is well characterized and is usable from now on. In order to verify the efficacy of the protein according to the invention in counteracting or destroying the above-mentioned microorganisms, various experiments were set up, in which the protein, in various purified and non-purified conditions, was placed in contact with representative bacteria, fungi, phytoplasmas. and with the spike protein produced by Sars Cov-2.

S. aureus.

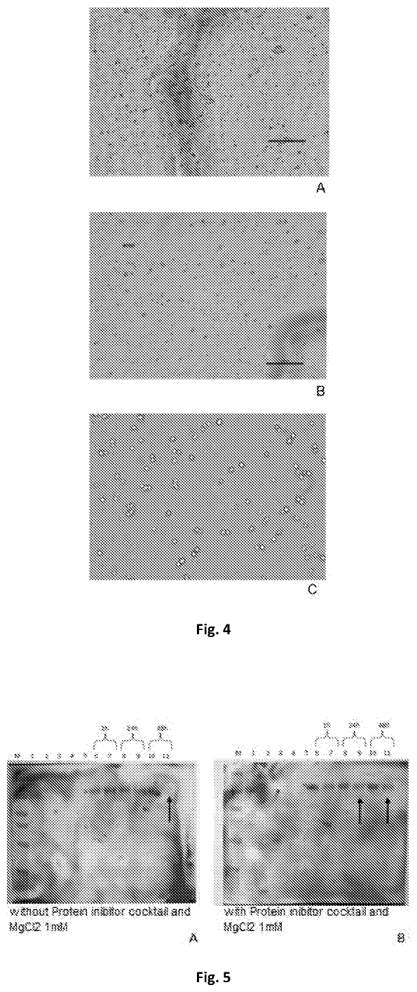

FIG. 4 shows an experiment in which the crude extract, at a concentration of 1 μg, was added to a bacterial culture of aureus subsp. aureus (atcc 6538P) at a concentration of 1000 bact/ml and the possible biocidal effect was observed at 48 and 72 hours.

The purpose of this experiment is to observe possible antibacterial effects. As can be seen from the photos represented in FIG. 4 compared to the initial culture (represented at TO in panel C), a progressive destruction of the bacterial bodies is observed with the production of microscopic gas bubbles generated by the production of H++ and O22—. In particular, FIG. 4a shows an optical microscope photo of a culture of S. aureus coagulase positive after contact for 48 hours with the crude extract of the protein, in FIG. 4B the same culture is represented at 72 h from the contact.

It can be seen that the concentration of microorganisms has decreased by 80% and an increase in gas bubbles generated by the lysis reaction is observed. The surface of the plate therefore has a total absence or very little presence of gram+bacteria thanks to the reducing effect given by PGIP+GTF1.

Aspergillus

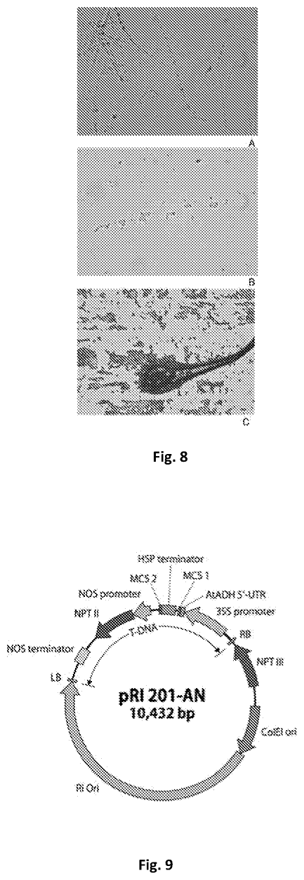

FIG. 8 shows an experiment in which the raw extract was added to a culture of Aspergillus (ATTC16404) and the possible fungicidal effect was observed 72 hours after contact. FIG. 8C shows the photo of a microscopic mushroom with its hypha at time zero. FIG. 8A shows the lysis effect of the entire hypha after 72 h from contact with the raw extract and the relative magnification is shown in photo 8b. The experiment demonstrates that the raw unpurified fusion protein has a fungicidal effect.

Sars-Cov2

In order to verify the effectiveness of the protein according to the invention in fighting viruses, an experiment was set up (FIG. 5), in which the protein according to the invention was placed in contact with the spike protein produced by SARS-CoV-2.

The protein according to the invention was purified, extracted with or without PMSF and E-64 protease inhibitors (respectively serine-protease and cysteine-protease); both extracts with the addition of 10 mM of MgCl2, as magnesium acts as a co-enzymatic activator, were placed in contact with a mixture of sars-Cov2 spike proteins (abcam) in a ratio of 5:1; the protein according to the invention is present at the final concentration in 1 μg/μl.

The result of this contact was observed after 1, 24, 48 hours, with relevant effects.

In FIGS. 5a and 5b, western blots performed after gel run in SDS-PAGE are represented. Membranes were incubated with anti-spike antibodies. FIG. 5A shows the result of the experiment conducted without protease inhibitors, FIG. 5B shows the result of the experiment conducted with protease inhibitors.

In lanes 6 to 11 it is possible to observe the progression of the experiments at different contact times. In FIG. 5a, in the last lane (11) indicated by the arrows, the absence of the spikes at 48 h of T with respect to the respective control (10) is noted. In FIG. 5b, on the other hand, in which the same experiment is repeated, but with protease inhibitors, it is possible to observe a decrease in the amount of spike proteins.

The arrows indicate the decrease in the intensity of the band already at 1 h, 24 h and 48 h. This experiment demonstrates that the purified protein in its 62 kDa non-glycosylated form possesses marked antiviral properties.

The result of this experiment demonstrates a mild effect of the initiation of protein lysis against the spikes of covid-19 already at 1 h attesting the virucidal activity of the protein already at the concentration of 1 μg/μl.

Cytotoxicity Experiments

To understand if a biological use of this fusion protein was possible without causing damage to human cells, a biocompatibility test was carried out to verify a possible cytotoxicity of the protein according to the invention and to understand the optimal concentrations of use.

Two cell lines were used to do this: human ovarian cancer cell line A2780 and lung mesothelioma cell line MSTO-211 H. The cells in the respective culture media are kept in an incubator in a humidified atmosphere at 37° C. and manipulated using a sterile laminar flow hood and incubating the cells at 48 and 72 hours in plates.

The cytotoxicity of two solutions was evaluated:

a) a solution containing the fusion protein at different scalar concentrations (concentration of the stock solution equal to 1.9 nM) and
b) a solution containing only the buffer of the fusion protein solution. 25,000-30,000 cells per well (for 72 h assays) or 50,000 cells per well (for 48 h assays) were placed in contact with 5 different protein concentrations from 1 nM up to 20 nM in order to obtain a Gaussian distribution of the data. It was decided to use immortalized cell lines because they are more stable and therefore more responsive and not subjected to the cell decay to which cell lines derived from the skin are subject.

If non-immortalized cell lines had been adopted in this experiment, normal cell decay would have led to a false count, which would have had to be compensated for by an inverse logarithmic factor and this would have led to an incorrect calculation, compared to a stable cell line.

Since the purified protein used in this experiment resulted from a purification process on an IMAC purification column, in an imidazole gradient from 10 to 100 mM, and from a subsequent denaturation step in guanidium isothiocyanate, to characterize its nature, the protein has been subjected to a purification process by means of a dialysis cassette with a purification buffer at two different osmotic pressures, generated by two different internal/external osmolarities, this internal/external concentration difference causes the salts to be extracted from the site of the fusion protein by purifying it. This solution is also used as a control to obtain the results of the test which takes into account the dialysis pad. The results obtained, calculated as a percentage of viability with respect to the control condition (for the 20 nM condition the viability values were also calculated with respect to the control condition+buffer and indicated with *) are shown in the following tables:

TABLE 1

| A2780 (human | % viability at 48 h (mean of two | | viability at 72 h (mean of two | |
|---|---|---|---|---|
| ovarian carcinoma) | duplicate experiments) | | duplicate experiments) | |
| Control | 100% | | 100% | |
| Control + buffer | 49% | 100% | 50% | 100% |
| Protein sol. 1 nM | 88% | | 82% | |
| Protein sol. 2.5 nM | 90% | | 83% | |
| Protein sol. 5 nM | 94% | | 86% | |
| Protein sol. 10 nM | 95% | | 103% | |
| Protein sol. 20 nM | 45% | *91% | 50% | *100% |

*Value calculated with respect to the control + buffer condition, considered 100%

TABLE 2

| MSTO-21 IH (human | | | viability at 72 h | |
|---|---|---|---|---|
| biphasic | | | (mean of two | |
| mesothelioma) | % viability at 48 h | | duplicate experiments) | |
| Control | 100% | | 100% | |
| Control + buffer | 38% | 100% | 64% | 100% |
| Protein sol. 1 nM | 89% | | 89% | |
| Protein sol. 2.5 nM | 97% | | 83% | |
| Protein sol. 5 nM | 119% | | 100% | |
| Protein sol. 10 nM | 119% | | 100% | |
| Protein sol. 20 nM | 29% | *16% | 56% | *89% |

*Value calculated with respect to the control + buffer condition, considered 100%

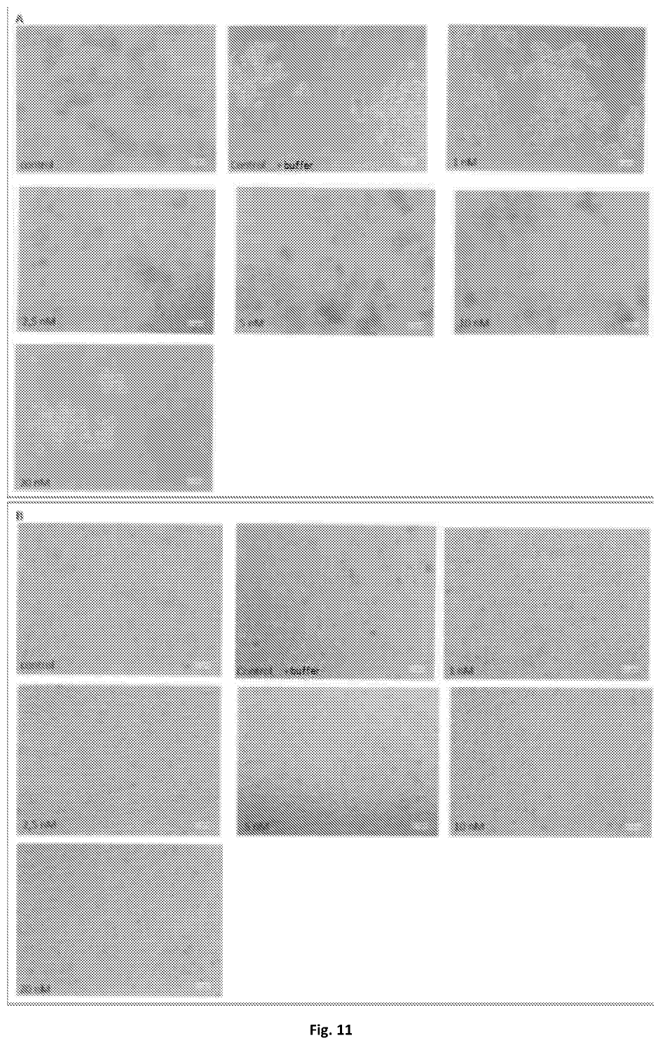

The results of these experiments are also represented in FIG. 11 where it is possible to observe the cell viability at 72 h for the different protein concentrations used for both cell lines in particular panel A A2780 and panel B MSTO-211 H.

As regards the results obtained in table 1 (A2780), the experiment considers a cut-off of 50% calculated on the two different immortalized cell lines. It has been shown that net of the cut off caused by the buffer, the mean survival of the A2780 cell line shows that at the protein concentration of 5 nM the mean cell survival has only a difference of 8% between 48 and 72 h. While at a concentration of 10 nM the average survival increases by 8%. Demonstrating that the toxicity of the protein net of the cut-off is compatible with cells at concentrations between 5 nM and 10 NM.

In the MSTO211 H cell line it is noted that at the concentration of 5 and 10 nM there is a trend reversal where at 48 h the survival increases by 19%, while at 72 h it normalizes. In this case the two protein concentrations on this cell line are not toxic, see table 2 (MSTO211 H).

The maximum toxicity is demonstrated in tables 1,2, they are compensated by comparing them with the cut-off of 50% at the maximum concentration of 20 nM, where a mortality rate of 11% is noted, in table 2, compensated by the cut-off of the 50% which maintains cell survival at 89%.

In table 1 the same, at the concentration of 20 nM there is a decrease of the same of 11%, this data is always compensated with reference to the cut-off of the control cells control+buffer.

It is therefore possible to conclude that the fusion protein according to the invention does not exhibit relevant cytotoxic effects on human cell cultures in vitro.

Agroadhesion and effect on mycoplasma/phytoplasmas and parasitic fungi

Mycoplasma are a class of microorganisms completely devoid of cell walls. Thanks to this characteristic they are completely immune to penicillins and to all those antibiotics that act on the cell wall biosynthesis process (such as cephalosporins). Their cell membrane has also evolved in order to compensate for the lack of the peptidoglycan wall: in fact its composition is very particular and different from that of other microorganisms; it is rich above all in sterols (unique case among bacterial species) and this allows them to keep their cell volume constant and resist water stress. Mycoplasma can be pathogenic to humans, animals and plants; the pathogenic mycoplasmas of plants are commonly divided by botanists and agrarians into two large classes, regardless of the species: Spiroplasmas (spiral-shaped and cultivable in vitro) and Phytoplasmas (which have variable shape, are completely obligate parasites and are not cultivable in vitro); both the ones and the others are in any case always Mollicutes and therefore have all the characteristics listed above, as well as a few other typical ones.

In order to verify the effectiveness of the PIGP+gtf1 protein in countering mycoplasma infection or infestation, a mixture of raw Pichia Pastoris extract as defined above and containing the PGIP+1GTF1 protein with a non-ionic tackifier was used directly on the leaves. (poly-1-pmenthene, glycolic extracts, ethoxylated isodecyl alcohol) at the concentration 0.005%-0.0025%. Specifically, heptamethyltrisiloxane modified polyalkylene oxide was used with a dilution of 0.005%; said vehicle has proved to be surprisingly effective here for anchoring the propagated molecule on plant surfaces, placed in a vertical position.

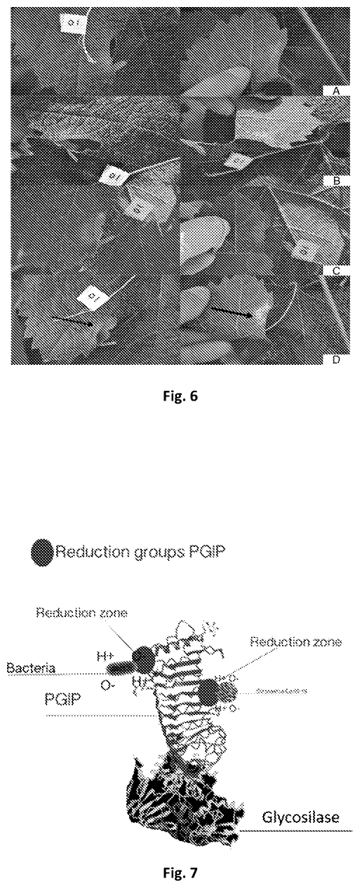

The photos shown in FIG. 6 ABCD describe the blocking effect of the infection of downy mildew (plasmopara viticola) on leaves of Vitis vinifera, at different times, by means of the soaking of the foliar adhesive used at the aforementioned concentration, the treatment allowed, the protein propagation. The purpose of this test demonstrates not only the effectiveness of the protein in agriculture and also the action against infestations of mycoplasma and in particular plasmopara viticola.

In FIG. 6 it is in fact possible to observe the progressive effect of the adhesion of PGIP+GTF1 on the leaves. In panel A it is possible to observe the leaves at time 0. The contact with the protein according to the invention is such as to cancel the diffusion and growth of plamopara viticola on the leaf surface in 10 hours (observable in panel 6B); with the progression of time it is possible to observe its consolidated blocking effect at 24 hours (panel C) and after 48 hours (panel D). The experiment demonstrates the progressive effectiveness of the anti-pest action of the PGIP+GTF1 complex after a single treatment.

This experiment demonstrates how phytoplasmas (microorganisms completely similar to mycoplasma) are effectively blocked after a few hours of contact.

The protein according to the invention therefore has proven antimicrobial efficacy, that is, it is capable of inhibiting the growth of bacteria, fungi, viruses and mycoplasma and phytoplasmas.

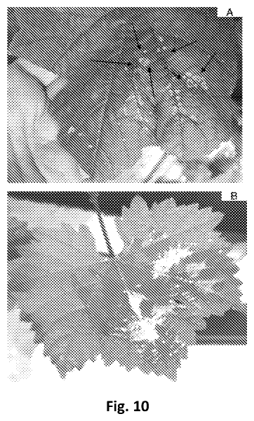

In another embodiment, the method of agroinfiltration of a suspension of Agrobacterium tumefaciens in the intercellular spaces of the leaves was used by spraying or by using a syringe without a needle; in fact it has been shown that good levels of transient gene expression are obtained with this method (Santos-Rosa et al. 2008; Zottini et al., 2008; Bertazzon et al. 2011.). In particular, the fragment of SEQ ID no 3 according to the invention is inserted in a vector suitable for use in A. Tumefaciens and subsequently in this electroporate; preferably the vector is the vector pRI-201AN (takara) which possesses two multiple Boning sites which therefore provides the vector with the ability to double express PGIP-GTF1, and the vector after purification is electroporated in the non-tumorigenic bacterium A. tumefaciens LB 4404 (takara). The A. Tumefaciens thus obtained are subsequently used directly on the leaf tissue, preferably a suspension of diluted product (400 μg) is made together with the non-ionic glue solution at a concentration of 0.0005%, and sprayed on the leaf. In FIG. 10 A we see a leaf infected with Botrytis cinerea, a fungus of the Sclerotiniaceae family, just agro-infiltrated at time zero and in FIG. 10b the effects of blocking the infection after 10 h from agro-infiltration. In this last photo we can see how the infection was quickly blocked and circumscribed.

The object of the present invention is therefore a composition comprising A. Tumefaciens transformed with an expression vector, comprising a nucleic acid having a sequence of SEQ ID no 3 or a sequence with at least 90% sequence identity with SEQ ID no. 3 or a sequence with at least 95% identity with SEQ ID no. 3 and at least one non-ionic tackifier or glue, preferably at 0.0005%.

Therefore, the protein according to the present invention can be defined as an antibacterial, antifungal, antiviral and disinfectant agent.

More specifically, the invention therefore relates to the use of the protein according to the invention for antimicrobial preparations for use in various fields.

Setting Medical and Veterinary

In particular, having regard to its safety demonstrated in the cytotoxicity experiments, it is object of the present invention a protein having amino acid sequence of SEQ ID No. 4 and/or a protein having 90% and more preferably 95% of identity sequence with SEQ ID no.4 for use in the medical field. Another object of the present invention is said protein for use as a medicinal product defined by its antimicrobial function, ie antiviral, antibacterial, antifungal, antifungal. The protein according to the invention can be used in compositions formulated in liquid form, as a cream or lotion or as a gel or spray for topical applications on animals and humans. Topical applications include applications on the skin and mucous membranes. The carriers can be all those used in the pharmaceutical and cosmetic fields. The adjuvants and carriers are those cosmetically and pharmaceutically acceptable, as well as the adjuvants and carriers used in the phytopharmaceutical field.

Carriers include lipid carriers, preferably single and multi-lamellar liposomes; in a preferred embodiment, the protein according to the invention is in fact packaged or encapsulated in said structures to allow more effective delivery to the treatment site, better diffusion and protection from self-oxidation, thus extending the time of residence on the surfaces, and promoting affinity with microbial structures.

The protein according to the invention is preferably administered topically, cutaneously and/or oropharyngeal-nasal and can be formulated in sprays, aerosols for inhalation, gels, creams and lotions. The object of the present invention is therefore a composition comprising the protein having SEQ ID no 4 and/or a protein having 90% and more preferably 95% sequence identity with SEQ ID no.4; optionally said composition comprises at least one of saline buffer, preferably PBS, protease inhibitor, MgCl₂, pharmaceutically acceptable excipients, carriers, thickeners and gelling agents. In a preferred embodiment, the composition according to the invention further comprises cellulose, preferably methylcellulose. The composition comprising the protein according to the invention can also be formulated in spray, semi-liquid, creamy, semi solid or solid forms, creams, suspensions, milks or soaps.

The composition according to the invention can also be composed of a lysate of microorganisms expressing the protein having SEQ ID no 4 and/or a protein having 90% and more preferably 95% sequence identity with SEQ ID no.4;

Environmental Disinfectant

The results of the in vitro experimentation shown in the figures have shown a remarkable antimicrobial activity of the protein according to the invention against various microorganisms, in particular Gram positive bacteria, fungi, mycoplasma and viruses.

The protein having amino acid sequence of SEQ ID 4 and/or amino acid sequence having 90% and more preferably 95% sequence identity with SEQ ID no.4 is therefore usable as an environmental disinfectant and antimicrobial, both in human, animal and vegetable fields. The protein according to the invention can be used alone or included in a composition further comprising vectors known to the skilled in the art and can be applied by spraying, formulated in gel or applied in solution. A composition comprising the protein according to the invention can therefore be formulated in spray, semi-liquid, semi-solid, solid, suspension, or gel form to be applied on the surfaces to be treated. The application can also be a spray. This composition can also be used as an antimicrobial functional base in the field of disinfection in various areas, for example in the clinic, hospital and domestic field, in high traffic environments, in filters of air inlet and extraction systems or air conditioners or of ATU treatment units, in environments used for food preparation. In general, in all those areas where it is necessary to adopt antimicrobial prophylaxis. By way of non-limiting example, compositions comprising the protein according to the invention can be used in household hygiene products as a disinfectant; in skin disinfectants, in soaps etc. ex. in the disinfection of the intact skin, for example in the disinfection of the hands in the preoperative phase; in hospital wards, against the transmission of nosocomial cross-infections; in disinfectants or community hygiene products (e.g. hotels, airports, schools, doctors' offices or dental offices); in the disinfection of surgical instruments.

The composition comprising the protein according to the invention can further comprise at least one of saline buffer, preferably PBS, protease inhibitor, MgCl₂, cellulose and methyl cellulose, gelling agents, preferably methyl orixane or alginates such as calcium or sodium alginate. The object of the present invention is therefore a method for the control or elimination of viruses, gram+bacteria, mycoplasmas, phytoplasmas, microscopic spore and oospore fungi, preferably S. aureus and Sars-CoV-2 from environments or surfaces where this method comprises the application on the surface or on parts thereof of at least one of—a lysate of microorganisms, Pichia Pastoris or Bacillus subtilis, expressing the protein having SEQ ID no 4 and/or a protein having 90% and more preferably the 95% sequence identity with SEQ ID no.4
a protein having SEQ ID no 4 and/or a protein having 90% and more preferably 95% sequence identity with SEQ ID no.4—compositions comprising called protein. From the tests carried out, the protein according to the invention resists 48-72 h on surfaces at ambient T.

Agritech

The results of the in vitro experimentation shown in the figures have shown a remarkable antimicrobial activity of the protein according to the invention towards various microorganisms, in particular Gram positive bacteria, fungi, mycoplasma and viruses. In particular, a considerable activity of the protein according to the invention has been found in contrasting pathogenic microorganisms of plants. Therefore, the subject of the present invention is the synthetic fusion protein having amino acid sequence of SEQ ID 4 and/or amino acid sequence having 90% and more preferably 95% sequence identity with SEQ ID no. 4 for the treatment of infections in plant species, preferably in the agricultural and phytopharmaceutical fields; the protein according to the invention can be used for the same purpose both used as such and by means of the agroinfiltration technique.

The protein can be used in compositions formulated in liquid form, or in lyophilized form.

The application can be performed with compositions comprising the carriers typically used for applications on plants.

In one embodiment, the use of a non-ionic glue is preferred, which has the function of impregnating the leaf plant tissue to allow certain pesticides to penetrate, in this case it has been used to make root both the raw Pichia pastoris extract expressing both the PGIP+GTF1 protein and the protein itself. The adjuvants and carriers are pharmaceutically acceptable, as are the adjuvants and carriers used in the phytopharmaceutical field.

Carriers include uni and multilamellar liposomes. The composition comprising the protein according to the invention can also be formulated in liquid, semi-liquid or gel form to be applied on the plants to be treated. The application can also be a spray. In one embodiment the protein according to the invention is comprised in a composition further comprising a non-ionic tackifier at concentrations ranging from 0.0005% to 0.00025%. Among the non-ionic tackifiers, poly-1-pmenthene, glycolic extracts, isodecyl alcohol ethoxylate are preferred, even more preferred is heptamethyltrisiloxane modified polyalkylene oxide preferably at a concentration of 0.0005%.

The composition comprising the protein according to the invention can further comprise at least one of saline buffer, preferably PBS, protease inhibitor, MgCl₂, cellulose and methyl cellulose, gelling agents, preferably methyl orixane or alginates such as calcium or sodium alginate. The composition according to the invention can also be composed of a lysate of microorganisms expressing the protein having SEQ ID no 4 and/or a protein having 90% and more preferably 95% sequence identity with SEQ ID no.4 A composition comprising the protein according to the invention can therefore be used with an antimicrobial function in the agricultural field and for the treatment of diseases of plants in culture or in ornamental plants. The object of the present invention is therefore a method for treating plant pathogens, where said method comprises the application on the plant or on parts of it of at least one of—a lysate of microorganisms, Pichia Pastoris or Bacillus subtilis, expressing the protein having SEQ ID no 4 and/or a protein having 90% and more preferably 95% sequence identity with SEQ ID no 4—a protein having SEQ ID no 4 and/or a protein having 90% and more preferably 95% sequence identity with SEQ ID no.4—compositions comprising said protein. From the tests carried out, the protein according to the invention resists 48-72 h on surfaces at ambient T. In an alternative embodiment, the protein according to the invention can be used in infection techniques with A. Tumefaciens. The object of the present invention is therefore a method for treating plant pathogens where said method comprises introducing in said plants an expression vector comprising a nucleotide sequence of SEQ ID no. 3 and/or a sequence having at least 90% and more preferably 95% sequence identity with SEQ ID no. 3 by agroinfiltration with A. Tumefaciens. The following examples are provided for the sole purpose of illustrating the invention and are in no way to be considered as limiting its scope.

### PROTOCOLS AND EXAMPLES

Cloning of pBE-s DNA and pGAPZ ALPHA A Pr1 201-AN vectors:
1) Gently mix fresh competent cells and transfer 100 μl to a polypropylene tube.
2) Add to the 100 μl of pR101-AN cells in quantities 510 ng.
3) Incubate in an ice bath for 30′.

### 4) Incubate at +42° C. for 43″.

5) Return to the ice bath for 1-2′.
6) Add the SOC medium, pre-incubated at +37° C. up to a final volume of 1 ml.
7) Incubate by shaking at 160-225 rpm for 1 hour at +37° C.
9) Plate on selective media, typically less than 100 μl for each 9 cm diameter plate.
10) Incubate overnight at +37° C.
11) Selection of the colonies and amplification of the same by incubation overnight at +37° C. in LB plate with the selective antibiotic for which the plasmid has the specific resistance kanamycin/ampicillin.

PURIFICATION of plasmids after cloning, primary and secondary after centrifugation of the liquid, 250 μl of resuspension solution are added to the cell pellet after 250 μl of Lysis solution and 350 μl of neutralization solution are mixed, then centrifuged at 14,000 RPM for 5 min. Subsequently, the content is placed in a purification column and centrifuged at 14,000 RPM for 1 min. Once the eluate has been discarded, 500 μl of “wash solution” are added twice.

Once the eluate has been discarded, place 50μl of the “elution buffer” in the column and the concentration of the purity of the plasmid is calculated on the spectrophotometer, which is preserved at 20° C.

Enzymatic cutting of pBE-s plasmids DNA pGAPZ ALPHA A

Multiple reaction of enzymatic digestion and linearization.

In a final volume of 20 μl, mix:

Buffer 10PGIP+GTF1 2 μL pBE-s DNA/pGAPZ ALPHA A Da in a quantity ranging from 0.2 to
1 μg Restriction enzymes as follows:

### 1 μL KpNI

### 1 μL XBAI

### Nuclease Free Water Qb

The reaction proceeds according to the protocol known to the expert in the field.
Enzymatic cutting and linearization IN PR 201-AN
Multiple reaction of enzymatic digestion and linearization.in a final volume of 20 μl:
MixBuffer 10PGIP+GTF1 2 μL DNA Pr pBE-s DNA/pGAPZ ALPHA A Da in a quantity ranging
from 0.2 to 1 μg Restriction enzymes as follows:

### 1 μL XBAI

### 1 μL NDEI

Nuclease free water qb

Ligation of the Products

In a final volume of 20 μl the following are mixed: DNA of the PGIP+GTF1 complex gene linearized as above Insert DNA from 10 to 100 ng, molar excess 3:1 compared to the DNAdeI Vector in use and the everything is left at room temperature for an hour.

Alternative method: 15 μL of daligare products are inserted using “IN FUSION HD CLONING” (takara) Master Mix, PGIP+GTF1+carrier in use, always in a proportion 3:1 and in a total of 20 μL of volume+Nuclease free water to taste All kept at +50° C. for 15′ min. The whole is inserted into E. coli STELLAR 0 DH5a cells for re-cloning.

Plasmid Purification

1) add 250NL resupension solution (Jet Plasmid Thermofisher gene) to the cell pellet.
2) 250 μl of Lysis solution
3) 350 μl of neutalization solution,
4) centrifuge at 14.000 RPM for 5′ min
5) recover the supernatant and insert 500 μl in the purification column
6) centrifuge at 14.000 RPM for 5′ min
7) 500 μl of Washing buffer to wash column centrifuge at 14.000 RPM for 1′ min repeating twice
8) add 50 μl of resuspension solution and centrifuge at 14.000 RPM for 2′ min. The purified plasmid is stored at −20° C.

Electroporation in Bacillus subtilis/Pichia Pastoris A. Tumefaciens

1. Place 1.5 ml tubes containing PIGP+GTF1 competent cells and electro-competent b. subtilis/Pichia Pastoris on ice. For Pichia Pastoris, after electroporation, the plamsmide pGapz alpha A is linearized with Avrll at +37° C. 15 min in 20 μl.
2. Add 6 μl (1 ng) of binary vector plasmid DNA to 20 μl of competent cells of PICHIA PASTORIS BACILLUS SUBTILIS and A. TUMEFACIENS mix gently.
3. Place the 0.1 cm electroporation cuvette on ice.

### 4. set the Gene Pulser II to 25 μF, 200 0 and 2-2.5 kV.*1

5. Transfer the cells and DNA prepared in step 2 to the electroporation and electroporation cuvette.
6. Remove the cuvette from the porator, add 1 mL of SOC*2 media and transfer to a 14 mL round bottom tube.
7. Incubate for 1 hour at 30° C., shaking at 100 rpm.
8. Plate 50-100 μl of cells on LB agar plates with 50 μg/ml kanamycin/10 μg zeocin (depending on vector)*3 and incubate for up to 48 hours at 30° C.
9. Amplify the colony in liquid LB with Kanamycin/10 μg zeocin at +30° C./+37° C. (depending on the vector).

Immunoblot

In order to verify the production of the pgip+gtf1 protein and its production site, the cell pellets of the modified bacteria were sonicated after purification on his-tag affinity column demonstrating that the presence of the protein is intra-cellular, measuring its spectro-photometrically concentration, during elution in the purification process amounting to 1 μg/μl. A HIS-TAG positive control such as albumin and a negative control in well 3, consisting of 10 μl of loading buffer and running buffer, are inserted in well n.2 10ul. A raw extract was placed in well n.4 The concentration of the purified protein in well n.5 was evaluated in 1 μg/μl. subsequently, characterization was carried out by electrophoresis FIG. 3A and by Immuno-blotting 3B. 10 μl of the extract is loaded into the well together with the negative well and positive controls in a precast gel (pharmacia) in a 5-10% acrylamide gradient, after mixing with 2 μl loading buffer (4% SDS 10% 2-mercaptoethanol 20% glycerol 0.004% bromophenol blue 0.125 M Tris-HCl pH 6.8) while the running buffer consisted of 25 mM Tris 190 mM glycine 0.1% SDS). The gel, after the electrophoretic run, was fixed and colored by immersion in the dye solution (625 mM coomassie brilliant-blue; 50% methanol; 10% acetic acid) for 30 min., Then, after photographic detection FIG. 3A, it is decoloured with the solution (50% methanol-10% acetic acid for 24 h) The western blot which was conducted at constant 100V in running buffer, for 70′ min resting it on nitrocellulose and subjected to 380 mA for 90′ min. To verify the end of the electro-transfer, the membrane was colored with a solution of Ponceau S (Sigma) and then decoloured with bidistilled water until the red color disappeared completely. The membrane was then incubated in 100 ml of saturation solution consisting of 1×PBS (pH 7.2:80 mM Na₂HPO₄; 20 mM NaH₂PO₄x 2H₂O; 100 mM NaCl); 0.1% Tween 20; 8 gr of dry milk) for 16-18 hours at 4° C.

After washing with 0.1X PBS and 0.1% Tween 20 (Sigma). The resulting membrane after the blotting run was incubated in a solution containing 5 ml of anti-HISTAG mouse monoclonal primary antibody (Ab-Cam) diluted in PBS 1:5000 at room temperature for 1 hour. It is then washed again washed with 5 ml of PBS-Tween 20 solution, six times for 5′ min. and incubated with a further 5 ml of 1:10,000 diluted secondary antibody (rabbit anti-mouse conjugated with peroxidase, Sigma) at room temperature for 1 hour. After 6 washes with PBS-Tween 20 the proteins recognized by the antibody with the ECL method (Amersham) were visualized, following the instructions of the supplier company. The experiment confirms the presence of a purified protein in well 5 at about 62 Kda of molecular weight at a concentration of 1 μg/μl. While in well 4 the same hyper glycosylated protein at 200 Kda of weight is identified.

Plate contact with S. aureus and the raw extract and any other bacteria after 2 min sonication. at a frequency>20kH, on ice, the bacterial lysate is centrifuged for 30″ sec. in a 1.5 ml eppendorf type tube and centrifuged at 14,000 rpm for 10″ sec. The reading of the concentration of the raw extract reported a spectrometric reading at 595 nm, according to the Bradford method, obtaining the concentration of 400 μg. Subsequently 100 μl of supernatant after centrifugation of 1.5 ml at 1400 rpm for 2 minutes are placed in a Petri dish and allowed to react with a strain previously cultivated on selective medium for S. aureus coagulase positive calculating a final concentration of the raw extract of 100 μg. The contact between PGIP+GTF1 and s.aureus is left for 48 h FIG. 4A and FIG. 4b 72 h at room T, the result is observed under the microscope on a slide with respect to the zero contact time FIG. 4c. Where there is a marked difference in the concentration of bacteria.

Contact test with spike proteins and relative immunoblot

Spike proteins, ready to use (ab-cam) are left to react with the purified and isolated protein after being purified in a 10-200 mM imidazole gradient on an IMAC His-tag column. The reading of the purified protein concentration reported a spectrometric reading at 595 nm, according to the Bradford method, obtaining the concentration of 1 μg/μl. The experiment using the protein characterized in FIG. 3 was conducted in such a way as to have a solution of the spike proteins and the protein, according to the invention, in a 1:5 ratio. This mix is left to react for 1 h, 24 h and 48 h. These solutions in volume of 10 μl are then loaded into the wells of a precast gel (pharmacia) in a 5-10% acrylamide gradient, after mixing with 2 μl loading buffer (4% SDS 10% 2-mercaptoethanol 20% glycerol 0.004% blue of bromophenol 0.125 M Tris-HCl pH 6.8) while the running buffer consisted of 25 mM Tris 190 mM glycine 0.1% SDS). The gel was fixed and colored by immersion in the coloring solution (625 mM coomassie brilliant-blue; 50% methanol; 10% acetic acid) for 30 min., Then, after photographic detection, it is decoloured with the solution (50% methanol-10% acetic acid). The gel was then left for 24 hours in the bleaching solution to which 50% methanol-10% acetic acid was added). After the bleaching, the samples were run at constant 100V for 70′ min by placing them on nitrocellulose in running buffer and subjected to 380 mA for 90′. To verify the outcome of the electro-transfer, the membrane was colored with a solution of Ponceau S (Sigma) and then decoloured with bidistilled water until the red color disappeared completely. The membrane was then incubated in 100 ml of saturation solution consisting of 1×PBS (pH 7.2:80 mM Na₂HPO₄; 20 mM NaH₂PO₄×2H₂O; 100 mM NaCl); 0.1% Tween 20; 8 gr of dry milk) for 16-18 hours at 4° C. After washing with 0.1X PBS and 0.1% Tween 20 (Sigma). The membrane was incubated with primary mouse anti-spike-tag monoclonal antibody (Ab-Cam) diluted in PBS 1:5000 at room temperature for 1 hour. Then it is washed again washed with the PBS-Tween 20 solution, six times for 5′ min. and incubated with the secondary antibody diluted 1:10,000 (rabbit anti-mouse conjugated with peroxidase, Sigma) at room temperature for 1 hour. After 6 washes with PBS-Tween 20 the proteins recognized by the antibody with the ECL method (Amersham) were visualized, following the instructions of the supplier company.

Biocompatibility Experiments

Material Used:

A2780 cells (human ovarian cancer) are cultured in RPMI-1640 medium (Sigma Aldrich R6504) supplemented with 10% fetal bovine serum (Gibco). MSTO-211H (biphasic human mesothelioma) cells are cultured in RPMI-1640 medium (Sigma Aldrich R6504) supplemented with 2.38 g/L Hepes, 0.11 g/L Na-pyruvate, 2.5 g/L glucose and 10% fetal bovine serum.

The cells are kept in an incubator in a humidified atmosphere at 37° C. and handled using a sterile laminar flow hood. The cytotoxicity of two solutions was evaluated:

a) a protein solution (concentration of the stock solution equal to 1.9 micromolar) and
b) a solution containing only the buffer of the above protein solution.

Experimental Protocol:

For the treatment, the cells were seeded in complete medium in P24 breast plates under the following experimental conditions:
>25-30 thousand cells/well for the assays at 72 h
>50 thousand cells/well for the assays at 48 h.

After 24 hours from sowing the exhausted medium was removed and the cells were treated, in duplicate, according to the following scheme:

control (containing complete medium) control+buffer (containing complete medium added with the maximum volume of buffer used for the treatment and corresponding to the condition of maximum concentration of the protein, 20 nM) sol. 1 nM protein (complete medium containing the protein solution at 1 nM concentration) sol. 2.5 nM protein (complete medium containing the protein solution at 2.5 nM concentration) sol. 5 nM protein (complete medium containing the protein solution at 5 nM concentration) sol. 10 nM protein (complete medium containing the protein solution at 10 nM concentration) sol. 20 nM protein (complete medium containing the protein solution at 20 nM concentration) After 48 hours and 72 hours from the treatment the medium was removed, the cells were detached using a solution containing 10 mM trypsin and 0.3 mM EDTA in phosphate buffer and immediately counted under a light microscope using the vital trypan blue dye (0.1% (w/v) solution in phosphate buffer). The results obtained were calculated as a percentage of viability with respect to the control condition (for the 20 nM condition the viability values were also calculated with respect to the control condition+buffer.

Agro-adhesion/adhesion with plasmid and A. Tumefaciens

(Santos -Rosa et al. 2008; Zottini et al., 2008; Bertazzon et al. 2011). The coding sequence for the enzymes of interest PGIP+GTF1 was cloned into a pRI 201 AN (TAKARA) expression vector with which it is Agrobacterium tumefaciens was engineered by means of enzymatic cloning and electroporation techniques according to traditional methods. In a preferred embodiment, the hypervirulent, non-tumorigenic strain GV3101 of Agrobacterium in particular LB 4404 (takara) was chosen. occurs with more efficiency, as the vector pR 201-AN expresses the PGIP-GTF1 gene in double measure without the formation of galls, in a very short time span. Use of heptamethyltrisiloxane, modified polyalkylene oxide, (silwet velonex) impregnates the leaves very quickly and the addition of agrobacterium tumenfaciens increases its effectiveness; the effect of the adhesive in fact consolidates the lesion and makes the modified rhizome penetrate very quickly, allowing the immediate action of the construct, which begins to block the pathology.

### BIBLIOGRAPHY

Ausubel, F M, Brent, R., Kingston, R E, Moore, D D, Seidman, J G, Smith, J A, and Struhl, K. (1994) Current Protocols in Molecular Biology, Greene Publishing Associates and Wiley-Interscience, New York
Baron, M., Reynes, J P, Stassi, D., and Tiraby, G. (1992) A Selectable Bifunctional b-Galactosidase: Phleomycinresistance Fusion Protein as a Potential Marker for Eukaryotic Cells. Gene 114, 239-243
Brake, A J, Merryweather, J P, Colt, D G, Heberlein, U A, Masiarz, G R, Mullenbach, G T, Urdea, M S, Valenzuela, P., and Barr, P J (1984) a-Factor-Directed Synthesis and Secretion of Mature Foreign Proteins in Saccharomyces cerevisiae. Proc. Natl. Acad. Sci. USA 81, 4642-4646
Calmels, T., Parriche, M., Burand, H., and Tiraby, G. (1991) High Efficiency Transformation of Tolypocladium geodes Conidiospores to Phleomycin Resistance. Curr. Genet. 20, 309-314
Cregg, J M, Barringer, K J, and Hessler, A Y (1985) Pichia Pastoris as a Host System for Transformations. Mol. Cell. Biol. 5, 3376-3385
Cregg, J M, Madden, K R, Barringer, K J, Thill, G., and Stillman, C A (1989) Functional Characterization of the Two Alcohol Oxidase Genes from the Yeast, Pichia Pastoris. Mol. Cell. Biol. 9, 1316-1323
Drocourt, D., Calmels, T P G, Reynes, J P, Baron, M., and Tiraby, G. (1990) Cassettes of the Streptoalloteichus hindustanus ble Gene for Transformation of Lower and Higher Eukaryotes to Phleomycin Resistance. Nucleic Acids Res. 18, 4009
Evan, G I, Lewis, G K, Ramsay, G., and Bishop, V M (1985) Isolation of Monoclonal Antibodies Specific for c-myc Proto-oncogene Product. Mol. Cell. Biol. 5, 3610-3616
Gatignol, A., Durand, H., and Tiraby, G. (1988) Bleomycin Resistance Conferred by a Drug-binding Protein. FEB Letters 230, 171-175
Gietz, R D, and Schiestl, R H (1996) in Methods in Molecular and Cellular Biology, in press
Henikoff, S., and Cohen, E H (1984) Sequences Responsible for Transcription Termination on a Gene Segment in Saccharomyces cerevisiae. Mol. Cell. Biol. 4, 1515-1520
Imiger, S., Egli, C M, and Braus, G H (1991) Different Classes of Polyadenylation Sites in the Yeast Saccharomyces cerevisiae. Mol. Cell. Bio. 11, 3060-3069
Kozak, M. (1987) An Analysis of 5AL-Noncoding Sequences from 699 Vertebrate Messenger RNAs. Nucleic Acids Res. 15, 8125-8148
Kozak, M. (1990) Downstream Secondary Structure Facilitates Recognition of Initiator Codons by Eukaryotic Ribosomes. Proc. Natl. Acad. Sci. USA 87, 8301-8305
Kozak, M. (1991) An Analysis of Vertebrate mRNA Sequences: Intimations of Translational Control. J. Cell Biology 115, 887-903
Mulsant, P., Tiraby, G., Kallerhoff, J., and Perret, J. (1988) Phleomycin Resistance as a Dominant Selectable Marker in CHO Cells. Somat. Cell Mol. Genet. 14, 243-252
Perez, P., Tiraby, G., Kallerhoff, J., and Perret, J. (1989) Phleomycin Resistance as a Dominant Selectable Marker for Plant Cell Transformation. Plant Mol. Biol. 13, 365-373
Sambrook, J., Fritsch, E F, and Maniatis, T. (1989) Molecular Cloning: A Laboratory Manual, Second Ed., Cold Spring Harbor Laboratory Press, Plainview, N.Y. Scorer, C A, Buckholz, R G, Clare, J J, and Romanos, M A (1993) The Intracellular Production and Secretion of HIV-1 Envelope Protein in the Methylotrophic Yeast Pichia Pastoris. Gene 136, 111-119
Waterham, H R, Digan, M E, Koutz, P J, Lair, S V, and Cregg, J M (1997) Isolation of the Pichia Pastoris Glyceraldehyde-3-Phosphate Dehydrogenase Gene and Regulation and Use of its Promoter. Gene 186, 37-44
Zaret, K S, and Sherman, F. (1984) Mutationally Altered 3AL Ends of Yeast CYC1 mRNA Affect Transcript Stabilityand Translational Efficiency. J. Mol. Biol. 177, 107-136 Thi Lan Than Bien et al. J.Gen. Appi. Microb.175-182 (2014)

1. Nucleic acid encoding a synthetic fusion protein and comprising at least one of: a sequence of SEQ ID no 3, a sequence having at least 90% sequence identity with SEQ ID no. 3 or a sequence having at least 95% identity with SEQ ID no. 3.

2. A vector, comprising the nucleic acid according to claim 1.

3. The vector according to claim 2 wherein the nucleic acid is operably linked to a promoter sequence.

4. A synthetic fusion protein having Camino acid sequence of SEQ ID 4.

5. A synthetic fusion protein having amino acid sequence with at least 90% sequence identity with SEQ ID no.4.

6. Antimicrobial composition comprising the protein according to claim 4.

7. The antimicrobial composition according to claim 6 further comprising at least one of, salt buffer, PBS, protease inhibitor, MgCl₂, excipients, carriers, thickeners, gelling agents, methyl orixane, calcium alginate, sodium alginate, carriers, adjuvants, cellulose or methylcellulose.

8. Composition comprising the protein according to claim 4 and a lysate of bacterial or fungal cells.

9. The antimicrobial composition according to claim 6 formulated in spray, semi-liquid, semi-solid, solid, suspension, or gel form.

10. Protein according to claim 4 for use as at least one of: a disinfectant or antimicrobial agent in, outpatient, hospital or home settings, in the disinfection of surgical instruments, in high traffic environments, on surfaces and handles, in filters of air inlet and extraction systems, in air conditioners, in air treatment units, in environments used for food preparation, or in the agricultural field.

11. Protein according to claim 4 for use in skin disinfection.

12. Protein according to claim 4 for use in the control and elimination of; viruses, gram positive bacteria, mycoplasma, phytoplasma, microscopic, spore fungi or oospore fungi.

13. Use of the protein according to claim 4 in a biological method to counteract the attack and proliferation of; viruses, gram positive bacteria, mycoplasmas, phytoplasmas, microscopic spore fungi or oospore fungi.

14. Method for the control or elimination of; viruses, gram positive bacteria, mycoplasma, phytoplasmas, microscopic spore fungi or oospore fungi from environments or surfaces wherein the method comprises application on the surface, or on parts thereof, of at least one of,
a lysate of microorganisms, expressing the protein having SEQ ID no 4 or a protein having 90% identity of sequence with SEQ ID no.4;
a protein according to claim 4; or
an antimicrobial composition comprising said protein.

15. Process for the production of a protein according to claim 4 comprising the following basic steps:
I. inserting in an expression vector, comprising a selection marker, a nucleic acid comprising at least one of: a sequence of SEQ ID no 3, a sequence having at least 90% sequence identity with the SEQ ID no. 3 or a sequence having at least 95% identity with SEQ ID no. 3;
II. using said vector for the transformation of competent cells suitable for the use of said vector;
III. selecting the competent cells transformed with said vector and multiplying the competent cells in culture;
IV. performing a lysis of the competent cells of step III; and
V. selecting and purifying the protein according to claim 4 from the lysate obtained in step IV.

16. The process according to claim 15, further comprising a step of encapsulating the protein obtained at step V in lipids.

17. The process according to claim 15, further comprising a freeze-drying step.
